(19) United States
(12) Patent Application Publication
(10) Pub. No.: US 2015/0073475 A1
(43) Pub. Date: Mar. 12, 2015

(54) Joint stability device and method

(71) Applicant: Michael Paul Schaller, Redwood City, CA (US)

(72) Inventor: Michael Paul Schaller, Redwood City, CA (US)

(21) Appl. No.: 14/121,505

(22) Filed: Sep. 12, 2014

Related U.S. Application Data
(60) Provisional application No. 61/876,758, filed on Sep. 12, 2013.

(51) Int. Cl. A61B 17/04; A61F 2/08

(52) U.S. Cl. CPC A61B 17/0401; A61F 2/0811; A61B 2017/0427; A61B 2017/0441; A61F 2002/0817; A61F 2002/0852
USPC 606/232

(57) ABSTRACT

An apparatus for stabilizing a joint during orthopedic surgery. The apparatus includes a securing anchor which is rigidly attached to a bone, and a flexible cable which is connected to the anchor at a point which is external to the profile of the bone. The flexible cable is constructed of a substantially inelastic material such as stainless steel. The cable is connected to features and mechanisms which provide an apparent elasticity in the apparatus when the cable is placed in tension. Further the apparatus includes adjustment features and mechanisms which allow the user to adjust the amount of tension along the length of the cable. The bones may be the femur and tibia in the stifle joint of a dog.

### PRIORITY

This patent application claims priority from provisional United States patent application:

Application Ser. No. 61/876,758, filed Sep. 12, 2014, entitled, “Joint Stability Device and Method” naming Michael P Schaller as inventor.

### FIELD OF THE INVENTION

The invention generally relates to joint surgery. More specifically, the invention relates to a method and device for stabilizing a joint in a canine stifle joint.

### BACKGROUND

Injuries to ligaments are common in both humans and animals. In humans, there are roughly 250,000 surgeries to repair ACLs (anterior cruciate ligaments) each year. These injuries are typically caused by acute trauma where the ligament is over exerted and breaks. Treatment options generally consist of replacing the torn ACL with a new ligament either from the patient or from a cadaver. Canine's may have similar ligament injuries in their stifle joints, which is analogous to a knee joint. By contrast to humans, CrCL (cranial cruciate ligament) injuries occurring in canines may be caused by chronic over exertion of the ligament due to a ‘sliding drawer’ effect where the femur sits on the sloped plateau of the tibia. The cranial and caudal cruciate ligaments prevent the knee from sliding back and at times bear significant loads in doing so. Over time, these ligaments can become weak and eventually break which may allow the knee to slide posteriorly onto the meniscus causing other injuries and eventually lameness. There are an estimated 1 million surgical procedures in dogs to address CrCL injuries. A variety of surgical procedures, techniques, and devices have been developed to address CrCL injuries, mainly focused on improving joint stability.

The traditional method of repair, sometimes called an extracapsular repair or Modified Retinacular Imbrication Technique (MRIT), acts to replaces the torn CrCL with a suture which is wrapped around the fabellar bone and then passed through a hole which is drilled in the tibial tuberosity and held in place with metal clips. This method is primarily used for smaller dogs. It is generally accepted that the suture will provide stabilization to the joint for a period of 2-12 months while scar tissue forms, but will break afterwards.

A device called the TightRope, developed by Arthrex, may be also used. In this procedure a hole is drilled in the femur and the tibia, and the TightRope suture device is fed through the holes to hold the knee stable. The device is held in place with buttons on the ends of the holes. As with the MRIT, it is generally accepted that the suture loosens or breaks over time and may not provide stabilization for the remainder of the dog's life.

In another technique called the Tibial Tuberosity Advancement (TTA), the surgeon cuts the tibial tuberosity until it is separated from the tibia and then uses a plate and a spacer to shift anteriorly the location where the patella tendon connects to the tibial tuberosity. This changes the angle at which the patella tendon pulls on the femur and may prevent the femur from sliding down the tibia plateau.

In another technique called the Tibial Plateau Leveling Osteoplasty, the crown of the tibia is cut so that it is separated from the rest of the tibia and then rotated such that the plateau angle is reduced or eliminated between the femur. A metal plate is screwed into the tibial plateau and the tibia to hold it in place. This is the preferred technique for larger dogs.

Additional techniques such as the Tibial Wedge Osteotomy and the Triple Tibial Osteotomy exist.

### SUMMARY OF THE INVENTION

What is invented are improved methods and devices for providing stability to a joint. In various embodiments, an apparatus is described which includes a securing anchor which is rigidly attached to a bone, and a linking element which is connected to the anchor at a point which is external to the profile of the bone. The linking element described below may be a support wire or flexible cable constructed of a substantially inelastic material such as stainless steel. In some embodiments below, the cable may be connected to features and mechanisms which provide an apparent elasticity in the apparatus when the cable is placed in tension. In other embodiments, the apparatus may include adjustment features and mechanisms which allow the user to adjust the amount of tension along the length of the cable. In still other embodiments, the cable may be connected to a second anchor which is rigidly attached to a second bone wherein the cable imparts a force from the first bone to the second bone. The bones may be the femur and tibia in the stifle joint of a dog.

The general body of published research on the various methods of improving joint stability indicate that success is achieved at 6 months in 80-95% of dogs while the remaining dogs may continue to suffer from lameness.

In the procedures described above, especially those which use a suture or similar material to provide stability, several limitations exist. First these procedures may be difficult for the surgeon to adjust during or after the surgery to achieve the maximum potential benefit. For example, in the case of the MRIT procedure the surgeon uses metal clips to secure the suture through the bone. It may be difficult to apply the appropriate amount of tension to the suture while it is being clipped and then after it has been clipped it may be difficult to adjust the location of the clip on the suture. This may result in either too much tension or not enough tension in the suture. Similarly, the TPLO procedure requires screws to be drilled into the bone to secure a plate that holds the rotated crown of the tibia. Once the screws have been drilled and secure it may be difficult to reposition the crown of the tibia. Furthermore none of these procedures is easily adjustable after the surgical procedure. A method and device for stabilizing the knee that is adjustable would be beneficial.

Additionally, certain of these procedures may not have sufficient durability. A repeat injury may occur in dogs especially in procedures where suture is used to support the knee as it is accepted that the suture breaks or dissolves after a period of 2-12 months. A method and device for stabilizing the knee that is more durable would be beneficial.

Additionally, certain of these procedures may not have ideal compliance in the system. A system that has no compliance such as a very stiff rod may impart too much impact loading onto the joint. A system that has too much compliance such as a nylon suture may not support the joint sufficiently under heavy loading. A method and device that have an ideal amount of compliance would be beneficial.

In particular, these issues are observed with the extracapsular repair techniques, which represent a majority of CrCL repair procedures (such as the MRIT and Tight Rope procedures). The suture used for these procedures may not have sufficient strength to hold the high loads observed during routine activity. Normal walking loads on a CrCL can be estimated at SON depending on the weight of the dog, and high activity loads are estimated to reach 400-600N. These forces are a challenge for procedures requiring suture as a supporting element. First, the break strength of the suture may not be sufficiently high and as a result the suture may break once the dog resumes normal activity. Secondly, the suture may permanently deform and stretch under these loads and thereby no longer provide stability to the joint.

Additionally, extracapsular repair procedures typically use bone tunnels for the suture where the suture is passed through one or more tunnels and secured in the tunnel or at an opposite end of the tunnel. This presents further challenges because the suture is routed over a corner of bone as it exits the profile of the bone. Under high loads the bone may chip or otherwise degrade such that the suture may take a shorter path and the tension or support in the suture may be substantially reduced. Further the corner of bone may cause advanced degradation of the suture material. This is particularly evident in areas where the longitudinal axis of the bone tunnel is at a sharp angle from the exit profile of the bone or the tension path of the suture, as seen in bone tunnels at the femur. Stronger materials may be used in place of the suture such as woven stainless steel cable, which would substantially increase the strength and durability of the procedure, however this presents additional challenges. For example while flexible metal cables are longitudinally strong, they are more difficult to bend over tight radii. In particular bending a metal woven cable through a bone tunnel in the femur would present a tight radius such that the diameter of the metal woven cable would be required to be smaller than is desired for sufficient axial break strength. Additionally, while the strength of metal woven cables would be advantageous, their increased inelasticity as compared to suture materials may be challenging. Ligaments in the canine and human body include a certain elasticity which allows for small movements, like a spring. Metal woven cables may not have a similar elasticity.

Methods and devices which overcome these challenges would be useful.

### BRIEF DESCRIPTION OF THE DRAWINGS

FIG. 1 shows an isometric view of an exemplary embodiment of the present invention with a femur anchor and tibia anchor connected by a support wire.

FIG. 2 shows the apparatus of FIG. 1. with the stifle joint shown transparent such that the apparatus is fully visible.

FIG. 3 shows a section view of the tibia anchor of the apparatus of FIG. 1 which includes an adjustment mechanism

FIG. 4 shows a section view of the femur anchor of the apparatus of FIG. 1 which includes a compressively elastic element.

FIG. 5 shows an isometric view of another exemplary embodiment of the present invention with a femur anchor and a tibia anchor plate with an adjustment mechanism.

FIG. 6 shows an isometric view of another exemplary embodiment of the present invention with a femur anchor and tibia anchor which includes an adjustment mechanism external to the profile of the bone.

FIG. 7 shows a section view of the adjustment mechanism of FIG. 6.

FIG. 8 shows an isometric view of another exemplary embodiment of the present invention with a femur anchor and a tibia anchor, along with an adjustment mechanism along the length of the support wire between the anchors.

FIG. 9 shows a section view of the adjustment mechanism of FIG. 8.

FIG. 10 shows a front view of an implant system with a series of anchors

FIG. 11 shows a sectional view of a locking screw in the open configuration

FIG. 12 shows a sectional view of a locking screw in the locked configuration

FIG. 13 shows a sectional view of an anchor system with a looped support wire

FIG. 14 shows a sectional view of an anchor system with an extension spring element

FIG. 15 shows a sectional view of an anchor system with a tensioning mechanism

FIG. 16 shows a sectional view of an anchor system with an internal compression element

FIG. 17 shows a sectional view of an anchor system with an external compression element

FIG. 18 shows a sectional view of a support wire with a compression element along its length between the anchors.

FIG. 19 shows a sectional view of a support wire with an adjustment mechanism along its length between the anchors.

FIG. 20 shows a side view of an anchor system with a series of clamping anchors.

FIG. 21 shows an isometric view of a clamp anchor which is secure to a bone

FIG. 22 shows an isometric view of a clamp anchor with a ratchet mechanism

FIG. 23 shows a side view of an anchor system with a patellar spacer

FIG. 24 shows a side view of an anchor system with a standing eyehook

FIG. 25 shows an isometric view of a filament anchor system

FIG. 26 shows a sectional view of a filament anchor

FIG. 27 shows a sectional view of a notched clamping system

FIG. 28 shows a side view of an anchor system with a pivot support

FIG. 29 shows a side view of an anchor system with a femur support

FIG. 30 shows a front view of an anchor system with a stanchion support

FIG. 31 shows a side view of an anchor system with a patellar support

FIG. 32 shows a side view of an anchor system with a support wire guide

FIG. 33 shows a side view of an anchor system with a ligament connector

FIG. 34 shows an isometric view of the ligament connector shown in FIG. 33

FIG. 35 shows a side view of an anchor system with a temporary clamp

FIG. 36 shows a sectional view of the anchor system shown in FIG. 35 with a temporary clamp

FIG. 37 shows a sectional view of an anchor system with a clamp surface

### DETAILED DESCRIPTION AND OPERATION OF VARIOUS EMBODIMENTS

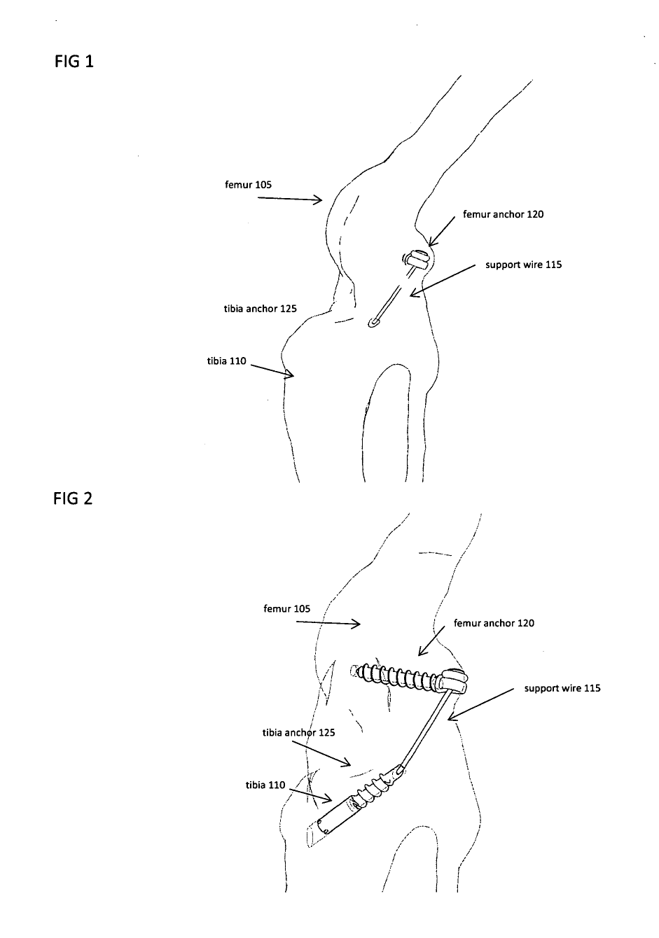

In FIG. 1 an exemplary apparatus is shown in an isometric view. A femur anchor 120 is shown extending from within the femur 105 and out of the bone profile. The femur anchor 120 has an eyelet or feature which is connected to the support wire 115 at a point which is external to the profile of the femur. A tibia anchor 125 is shown with the majority of the tibia anchor 125 within the profile of the tibia 110 as will be shown in FIG. 2. The support wire 115 is connected to the tibia anchor 125 and follows a path which may be considered similar to the patient's Cranial Cruciate Ligament (CrCL). In the following specification the term support wire is used interchangeable with the term linking element described within the claims of the invention.

In FIG. 2 the same apparatus of FIG. 1. is shown in situ within the anatomy, with the stifle joint transparent so the location and geometry of the apparatus may be seen. In this embodiment, the femur anchor has an external screw thread which may be advanced into the femur to the desired depth and rotation which allows the hole of the eyelet to be oriented at a desired angle relative to the path of the support wire. The tibia anchor 125 can be seen and may also have an external thread as shown rigidly connect the anchor to the tibia bone. In the embodiment shown the distal portion of the tibia anchor is threaded while the rest of the anchor is shown without a thread. The external thread may exist along just the distal portion of the anchor. Alternatively the external thread may exist along a proximal portion of the anchor, or may still exist along the entire length of the anchor. The external thread may be between 3 and 6 mm in diameter with a core diameter between 1.5 and 5 mm. The external thread may be a variable pitch or a fixed pitch between 1.5 and 4 mm. The support wire routes from the connection point on the femur anchor down and anteriorly across the stifle joint to a location on the tibia where the tibia anchor protrudes.

A critical feature of the embodiment shown is the external connection location of the support wire to the anchor. As can be seen from the FIG. 1 and FIG. 2 the angle of the femur anchor is quite acute relative to the angle of the support wire. Others have described anchor systems where the support wire is substantially aligned with the longitudinal axis of the anchor and the bone tunnel. In the case of extracapsular CrCL repair, the angle to enter the femur anchor or femur bone tunnel is quite acute. As described above this presents several challenges for maintain the tension within the support wire as well as maintaining the tension in the support wire. In the embodiment shown and others shown below, the point of connection of the support wire is external to the profile of the bone. This allows the support wire to connect to the femur anchor at an angle which is substantially in line with the path it is already traveling from the tibia anchor. The longitudinal axis of the support wire is not in line with the longitudinal axis of the femur anchor.

Furthermore the described methods may be advantageous for additional reasons. Certain extracapsular repairs such as MRIT and the TightRope procedure apply tension the suture or support wire while the canine is unconscious during surgery. This may pose challenges because additional slack may be present within the length of the suture especially at the areas of tight cable routing and particularly at the bone tunnel and suture interface. For example, the suture may appear to have sufficient tension to the surgeon during the intraoperative period, however once sufficiently high loads are placed on the suture such as during periods of high activity the suture may both stretch and orient into locations around the bone tunnel routing areas that have a shorter path length thereby reducing the tension within the suture. Furthermore the corners of the bone tunnels may degrade over time and round or chip away such that the suture may similarly have an effectively shorter path length around the curve. These challenges are overcome with the apparatus described herein. In the embodiment shown in FIGS. 1 and 2 the support wire does not route into the bone tunnel axis at the femur anchor and is instead connected externally to the profile of the bone. In this embodiment, the support wire will not face the challenge of path length changes that other extracapsular repair techniques face. Furthermore, the tibia anchor may be aligned such that the routing surface and cable path is both very gentle for the support wire and the surface is comprised of a sufficiently hard material such as stainless steel or titanium such that the path length does not change over time. In these embodiments, the user may adjust the tension within the support wire to the appropriate tension and have an increased likelihood that the tension will remain more constant than other extracapsular repair techniques.

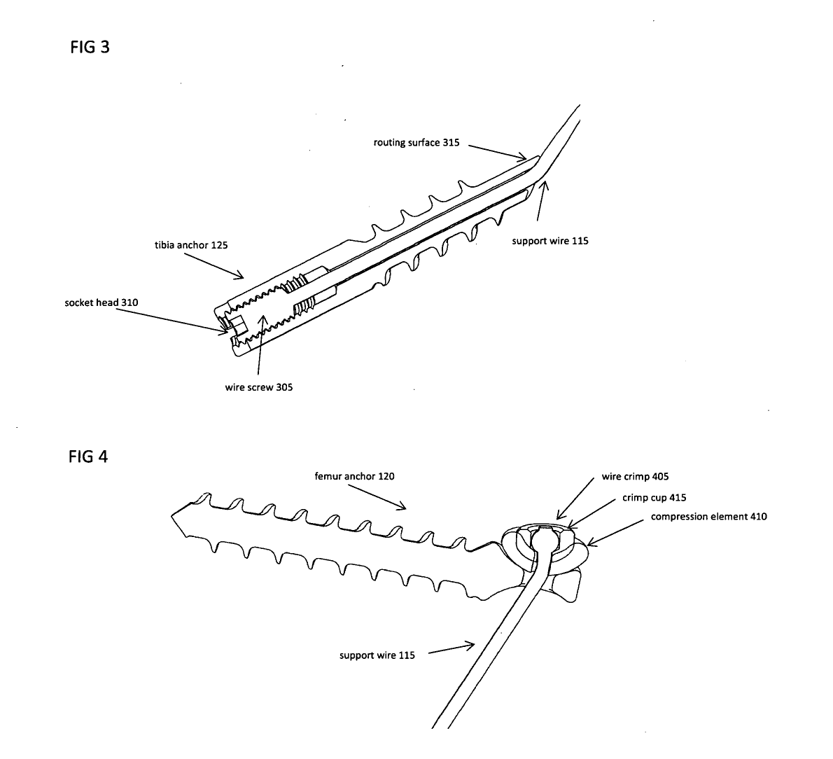

In FIG. 3 a close-up of the apparatus of FIG. 1 is shown with a section view along the length of the tibia anchor to show a possible mechanism within the anchor. The internal diameter may be hollow such that the support wire may pass through the length of the anchor. One end of the support wire may terminate at a crimp or other component within the anchor. In the embodiment shown, the support wire terminates and is held by the wire screw 305. The wire screw 305 may have external threads sized to be a #0 to #8 ANSI screw thread. The wire screw 305 may engage with corresponding internal threads along a least a portion of the internal lumen of the anchor. In the embodiment shown, the wire screw is at the proximal end of the anchor with a series of internal threads at the proximal end. In other embodiments, the internal threads may exist at the distal location of the anchor or along the entire length of the anchor. A potential advantage to placing the internal threads at the proximal end of the anchor and the external threads at the distal end is that the diameter of the wire screw may be larger than would be possible if it was to engage with internal threads which are smaller than the core diameter of the anchor. For example, in the embodiment shown the wire screw has external threads sized as a #4-48 thread and has an estimated pullout force between 150 and 300 lbf. The wire screw may have a socket head 310 which allows for adjustment of the position of the wire screw along the length of the anchor thereby pulling or imparting tension upon the support wire. Once the wire screw is adjusted to the desired location other mechanisms such as thread locking materials, locking pins, jam nuts, or any other suitable means for securing the wire screw may be used to hold the wires screw at a desired location. In a preferred embodiment, thread locking materials may be applied to the wire screw to prevent unintended movement of the wire screw.

In the embodiment shown the lumen of the anchor may be at least slightly larger than the outer diameter of the support wire which is shown as between 0.035″ and 0.060″. The support wire routes through the routing surface 315 at the distal end of the tibia anchor. The tibia anchor may be oriented within the tibia such that the support wire has a minimal angle of incidence between the tibia anchor and the femur anchor. The routing surface may provide a smooth surface with a gentle radius for the support wire to travel along. The surface may be polished or otherwise smooth to reduce the frictional force of the support wire on the routing surface and the deleterious effect on the support wire. The distal end of the tibia anchor may be beveled such that when it exits the profile of the bone, the anchor can be rotated to extend from the external profile of the bone as minimally as possible. Alternatively the anchor may be positioned such that the distal end and routing surface are appropriately aligned with the connection point of the support wire along the end of the femur anchor.

In FIG. 4. a section view of the femur anchor of the apparatus shown in FIG. 1 is shown schematically. The femur anchor as described above may have features which rigidly connect the anchor to the femur or other bone. In the embodiment shown, the connection features are an external thread which may be screwed or advanced into the profile of the femur to the desired depth. Alternative fixation methods may be contemplated such as bone cements, double pins, expandable features and any other suitable method for connecting the anchor to the bone. At least a portion of the femur anchor shown remains external to the profile of the bone and contains features which allow connection of the support wire. In the embodiment shown the support wire is held to the anchor by passing the support wire through the eyelet of the femur anchor and then placing a wire crimp 405 or other components such as a crimp cup 415 on the end of the support wire which is larger than the diameter of the eyelet. Other methods of connecting the support wire to the femur anchor may be contemplated such as slotted holes, wrapping pins, winches, and any other suitable means for connecting the support wire to the anchor.

Also shown in FIG. 4 is a compression element 410. The compression element is shown occupying a space between the support wire crimp and the connection point on the femur anchor. In this embodiment the compression element is a round elastomeric material with a through hole that the support wire travels through. As tension is imparted upon the support wire, the wire crimp and crimp cup are pulled down onto the compression element such that the compression element elastically deforms at a predetermined stiffness. The stiffness of the compression element may be adjusted to be similar to the stiffness of the natural ligament. For example, the compression element may have an outer diameter between 0.10 and 0.25″ with an internal lumen of 0.025 to 0.075″, and a thickness of 0.030 and 0.090″. The material may be a biocompatible polyurethane material with a modulus of elasticity of between 1000 and 20000 psi. In this embodiment, the spring rate k of the compression element may be between 500 and 3000 lb/in with the natural ligament spring rate estimated at 1350 lb/in. The adjustment mechanism described in FIG. 3 may be used to place a ‘pre-load’ upon the compression element between 2 and 40 lbf. This may allow the joint to be supported a variety of loading conditions and configurations. Alternatively, the compression element may be comprised of any other suitable material which may be compressed to provide a predetermined load at a given displacement. Elastomeric material such as silicones, thermoplastic elastomers, and the like may be considered. Alternatively, non-plastic materials such as nitinol may be considered.

Still other compression elements and dampening elements may be contemplated. The internal lumen of the anchor or the external connection point of the anchor may include a pneumatic or hydraulic element which dampens force applied to the support wire. Alternatively foams and any other suitable dampening materials may be considered appropriate.

In other embodiments, the external portion of the femur anchor may be deflected to provide an apparent elasticity in the support wire. For example, the femur anchor may be comprised of a flexible material or may be configured by a designed geometry, such as with cutouts, such that the eyelet of the anchor deflects substantially under a load. By deflecting the support wire length will have an apparent elasticity.

The support wire may provide stability to the stifle joint as the anchors can be fixated in locations which orient the support wire to provide a resistance force to the femur from moving posteriorly onto the meniscus or other tissue similar to other current stifle joint procedures such as MRIT or TightRope procedures previously described.

The anchor may be comprised of any number of materials. In an exemplary embodiment, a strong biocompatible metal such as a stainless steel like 316LL, 304, 302, or the like may be used. Alternatively, other biocompatible metals may be used such as titanium, nitinol, or the like. Similarly, other biocompatible plastics such as PEEK, polycarbonate, PTFE, or any other number of materials may be used.

The support wire may be comprised of any number of materials. In an exemplary embodiment, the support wire may be comprised of a braided metallic material such as a stainless steel like 316LL, 304, 302 or the like. Alternatively other metallic materials such as titanium, nitinol, tungsten or the like may be used. Furthermore, any number of non-metallic materials such as carbon fiber, Liquid Crystal Polymer (LCP), PEEK, or the like may be used. In an ideal embodiment the support wire may support a load above approximately 90-134 lbf (400-600N) at its highest load since this is the estimated peak tension applied to a CrCL during high activity (Caporn and Roe 1996; Wingfield et al. 2000; Burgess et al. 2010). A safety factor may be applied such that the peak load applied to the support wire never exceeds the ultimate strength of the support wire and that it has a high fatigue life. For example, if using a stainless steel material that is braided into a support wire with 7 filaments (commonly referred to as a 1×7 construction) the break strength of a 0.038″ support wire is estimated at 260 lbf. Alternatively, an even higher break strength support wire may be constructed from a 1×7 construction sized at 1/16″ diameter of 500 lbf. Further other constructions such as 1×19, 7×7, 6×19, or any other number of cable construction and sizes may be used from various materials to achieve an appropriate break strength. A benefit of a braided high strength material such as stainless steel is its high break strength for its relatively small size and high flexibilty. This addresses a major problem with existing stifle joint surgeries such as MRIT and TightRope procedures previously described, in that these procedures may use materials and single filament constructions that stretch or break over time from the relatively high loads imparted by the stifle joint. Alternatively a single filament support wire that is simultaneously flexible enough such as a single wire comprised of superelastic nitinol may be used because this may provide the high ultimate tensile strength required while still providing the essential flexibility of such a support wire. For example, superelastic nitinol may have a break strength of approximately 190 ksi. Therefore, a nitinol support wire that is 0.041″ in diameter would have an approximate break strength of 250 lbf. Alternatively a support wire comprised of a single filament which is notched in a pattern to provide high flexibility may be used. For example, while a stainless steel tube may provide high tensile break strength it may not be flexible enough to route from an anchor across a joint. In this case the tube may be cut with a laser, an EDM machine, or the like to create a series of grooves in the tube that allow the tube to bend easily.

In a description of the usage of the apparatus shown in FIG. 1, the user may pre-drill pilot holes into the femur and tibia bones as appropriate to allow the anchors to be screwed into the bones. The support wire assembly may come pre-attached to either or both anchors or no anchors. In the embodiment shown, the end of the support wire within the tibia anchor may be supplied to the user already connected to the wire screw with the other end free. After placing the anchors into the bones, the user may thread the free end of the support wire through the eyelet of the femur anchor and place a compression element and crimp cup as needed onto the length of the support wire. The user may then use a tool to crimp the wire crimp onto the support wire. The user may then tighten the support wire to the desired tension by moving the wire screw within the tibia anchor. At any point in the intra-operative or post-operative period the user may tighten or loosen the tension within the support wire by further adjusting the wire screw location. The user may use methods such as torque wrenches and torque tightening to properly adjust the tension in the support wire. For example, the user may deflect the support wire a given amount along its length between the anchors and measure the force or angle to determine the appropriate tension within the support wire.

During normal operation once the canine has recovered from surgery, the apparatus shall provide a force on the femur and tibia to assist in preventing the femur from sliding back. The force may increase at the stiffness defined by the apparatus and compression element as the amount of displacement is increased. Therefore at normal activity, the force may be between 2 and 40 lbf while the dog is walking. Under high loads a larger amount of tension may be imparted onto the support wire and compression element may deflect further. An advantage of a compression element over other elastic elements is that the compression elements tend to have higher failure forces. As described above and below the compression element may be a substantially elastic material whose stiffness is primarily dependent on the modulus of elasticity of the material and which returns to its original shape once the load is removed. Alternatively, other compression and elastic elements may be contemplated. For example, the compression element may be a compression coil spring that provides a spring force upon application of a load. Alternatively Belleville or disc springs may also be considered. In addition, tensile elastic elements may be considered which provide elasticity under tensile loads. For example, the support wire may include features which provide a predetermined elasticity through cable stretch, modulus of elasticity changes, and the like. Alternatively, the support wire may be comprised of multiple materials or components which have individual elasticity properties. For example the support wire may include a core section comprised of an elastic polymer while the external portion of the support wire may be a cable or tubing with substantially inelastic properties.

Other embodiments of an external connection between the support wire and anchors may be contemplated in the embodiments below.

In FIG. 5. an isometric view of the stifle joint is shown with an stability apparatus. The femur anchor is generally similar to the apparatus shown in FIG. 1. The tibia anchor however includes a similar external connection to the support wire. A tibia anchor plate is shown which is rigidly connected to the tibia on the outside surface of the bone with screws. The tibia anchor plate is oriented such that the plate adjustment screw is substantially at a location where the support wire may be routed around the plate adjustment screw. In this embodiment, the plate adjustment screw may be rotated to increase or decrease the length of the support wire and thereby increase or decrease the amount of tension applied to the support wire. The plate adjustment screw may include a bearing surface which is contained within the tibia anchor plate. When the plate adjustment screw is rotated the support wire is pulled along the circumference of the bearing surface such that the relative length of the support wire is increased or decreased until the desired tension is achieved. This may be accomplished intra- or post-operatively at any point. Additional mechanisms such as locking screws and pins may exist to lock the plate adjustment screw in place.

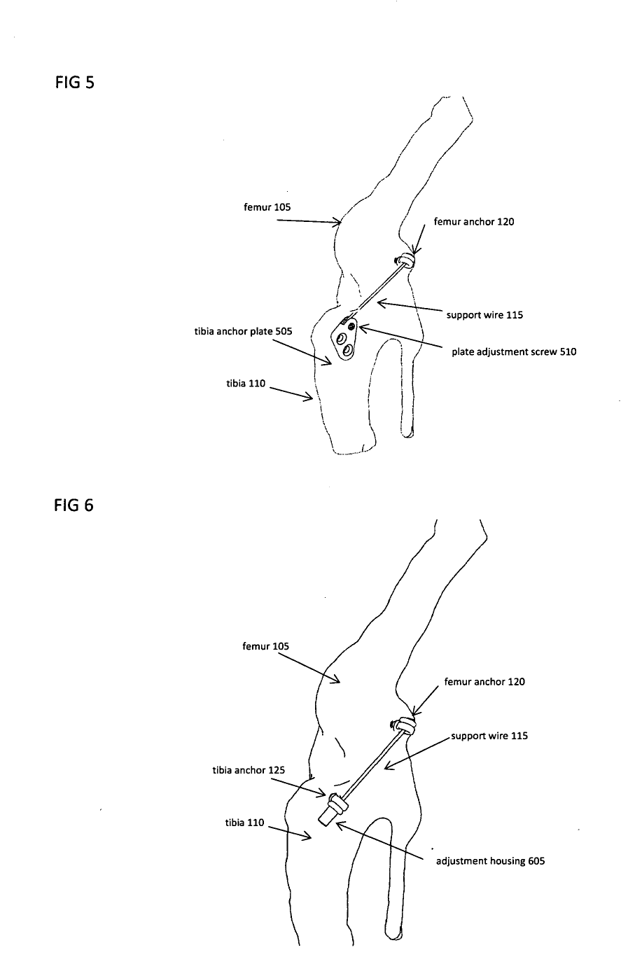

In FIG. 6 an isometric view of another apparatus embodiment is shown. The femur anchor and tibia anchor in this embodiment are similar in that both are secured to the bone with a portion of the anchor existing external to the surface of the bone. The tibia anchor may further include an eyelet or other attachment mechanism similar to the femur anchor for connecting the support wire. At one end of the support wire an adjustment housing may exist as shown in the embodiment in FIG. 6.

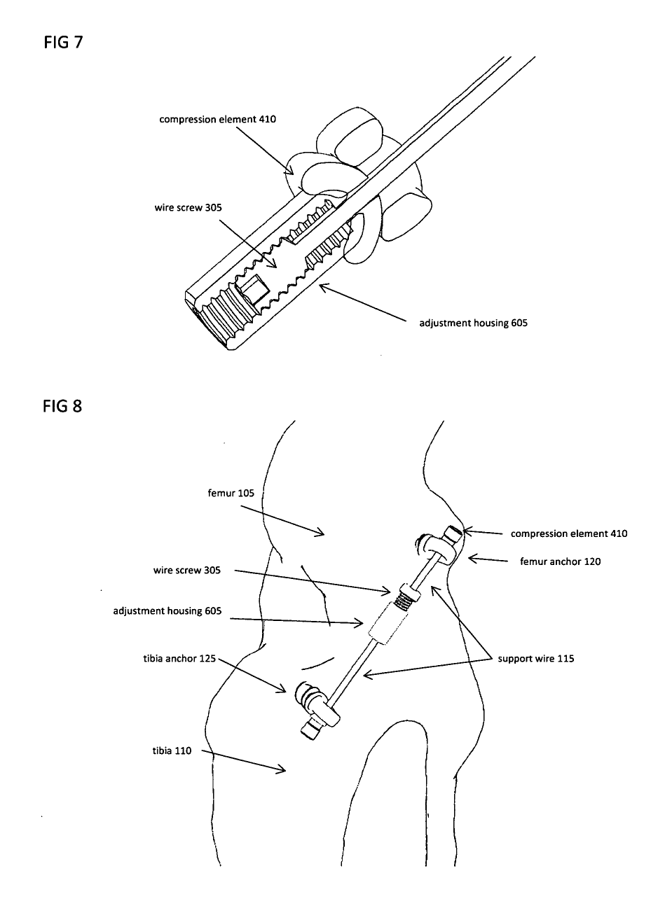

In FIG. 7 a close-up view of the adjustment housing is shown. The adjustment housing may have an internal or external mechanism for adjusting the tension of the support wire. In the embodiment shown the adjustment housing has an internal thread which engages with and external thread on the wire screw which is connected to the support wire. The user may rotate the wire screw into and out of the adjustment housing as needed to change the tension within the support tube. In the embodiment shown a compression element is shown at both the tibia anchor and the femur anchor. Likewise, the adjustment housing could exist at either one or both of the anchor ends.

In FIG. 8 an isometric view of another embodiment of the apparatus is shown. In this embodiment an adjustment housing and wire screw exist at a location along the length of the support wire between the two anchor connection points. The support wire may be comprised of two discrete lengths which are connected and adjustable at the adjustment housing as shown. Other methods of connecting the support wires may be contemplated and may include methods wherein the support wires are not coaxial or parallel. This may be advantageous because of the angles of the relative support wires from the anchors may not be aligned.

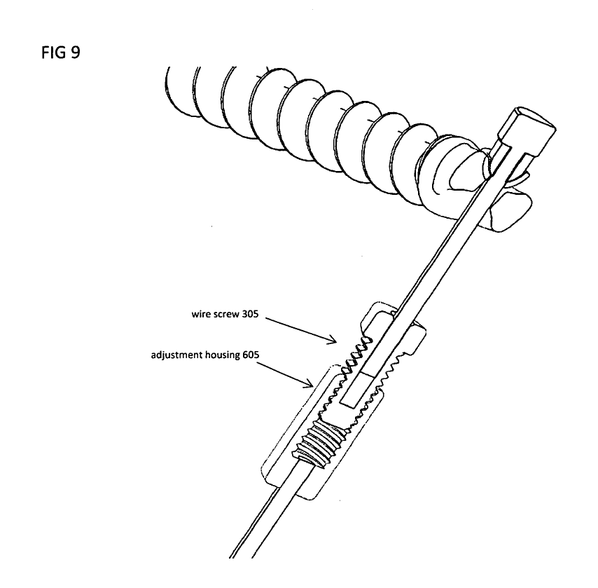

FIG. 9 shows a close-up view of the apparatus of FIG. 8 wherein the adjustment housing and wire screw are shown. In other embodiments shown below, the adjustment housing may further include a compression or elastic element which provides an apparent elasticity of the support wire when a tensile load is applied.

Still other embodiments and apparatuses may be considered. In descriptions below, alternate methods of connecting support wires, anchors, and other components are considered. These embodiments may or may not include points of connection between a support wire and anchor which are external to the surface of the bone. However, these embodiments should be considered in view of the claimed elements of this invention such that these embodiments may be additionally expanded to include such claimed elements.

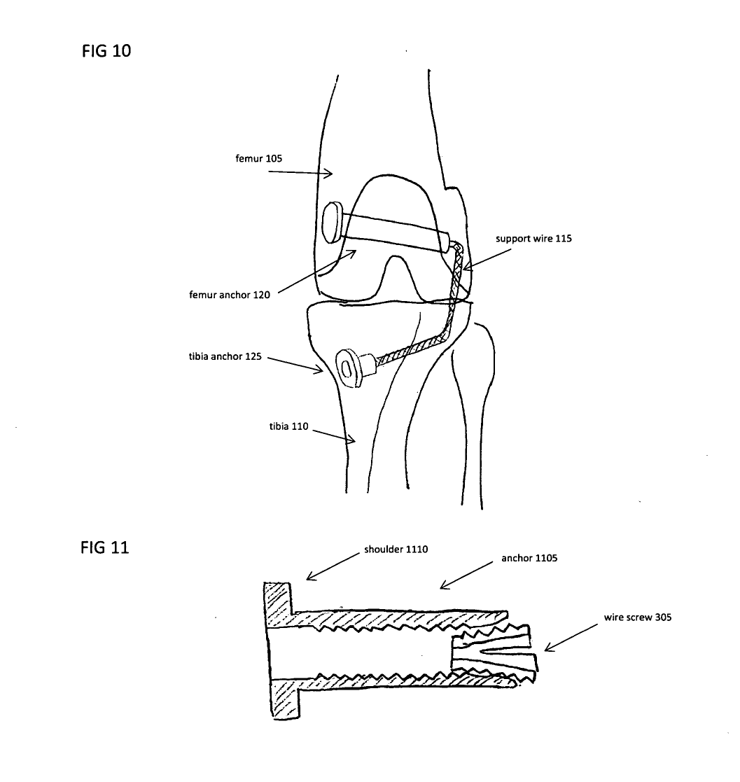

In FIG. 10 an exemplary implant system is shown from a front view of a stifle joint. In this embodiment a femur anchor 120 and a tibia anchor 125 may be used to secure a support wire 115. The femur anchor may be inserted into the femur after a hole has been drilled by the surgeon into the femur. The femur anchor may be inserted into the hole until the shoulder 1115 rests against the outside surface of the femur. Similarly the tibia anchor may be placed into the tibia with a shoulder resting on the outside surface of the tibia. These shoulders may provide a hard stop for the anchor such that tension may be applied to the support wire and the anchor will not move with regard to the bone. Alternatively, other methods of fixating the anchor may be implemented such as a threaded screw that is screwed into the bone. Alternatively, the anchor may be secured through the use of expanding features which expand within the bone to fixate it securely such as is used in suture anchors. Alternatively any other method of fixation such as bone cement or the like may be used to fixate the anchor. A support wire may be routed from one end of the femur anchor to another end of the tibia anchor. These termination points may be on the same side as shown (i.e. both on the lateral side or both on the medial side) or they may exist on opposite sides such that the support wire is routed through the stifle joint and to the other side as is the case certain types of extracapsular suture repairs. One anchor of the implant system may be adjustable as described in the embodiments below and one anchor of the implant system may not be adjustable. Alternatively, both anchors may be adjustable or both anchors may not be adjustable. In one embodiment, the femur anchor is adjustable since it may be a longer size due to the thickness of the femur at the site of implantation and the tibia anchor may be not adjustable. Alternatively, the tibia anchor may be adjustable and the femur anchor may not be adjustable.

FIG. 11 through 17 will now be described in greater detail which show anchor systems which connect a support wire to an anchor such as the tibia anchor or femur anchor. It should be appreciated that while certain embodiments are shown or described which demonstrate a variety of embodiments, additional embodiments exist which represent different configurations, combinations, and alterations on the described embodiments. For example, an anchor system that is described as positioned in the tibia may also be assumed to include similar anchor systems which are positioned in the femur.

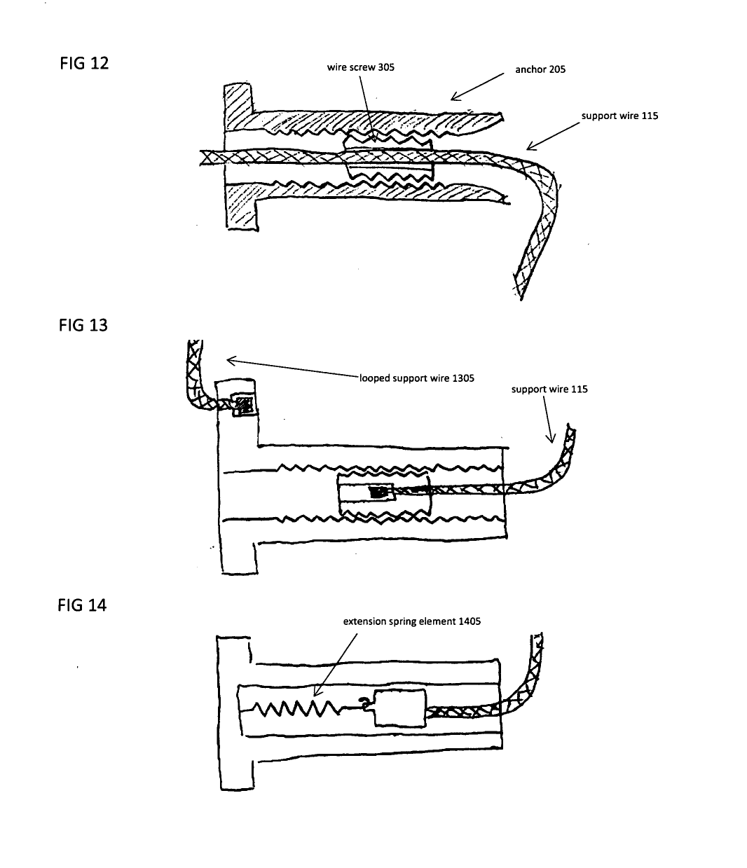

In FIG. 12 an exemplary embodiment of an anchor system is shown. The anchor may be an anchor that is attached to the femur or the tibia or any other location within the stifle joint or in any other location. The anchor may have a shoulder as described previously or may be attached to the bone in any number of other manners. In this embodiment, the support wire is connected a wire screw 305 within the anchor. The anchor may have internal threads and the screw may have external threads that engage with one another such that turning the screw advances the screw within the anchor. The screw may be comprised of any number of materials such as stainless steel, titanium, PEEK, or any other suitable material. The threads on the screw and anchor may be a standard pitch and diameter size such as a #4-40 which has a 1/40″ (0.025″) pitch. Alternatively other sizes and pitches may be used including custom pitches and variable pitches. The screw may have features on the side which is closer to the shoulder of the anchor which allow a tool such as an Allen wrench, square wrench, flat head screw driver, or the like to be inserted into the anchor and turned so that the screw is advanced axially.

The support wire may be connected to the screw in any number of manners. In an exemplary embodiment shown in FIG. 11, the screw may have slots at the distal end which flare the screw radially if it is not constrained within the anchor. This may allow a channel which runs partially through or entirely through the screw to be large enough for a support wire of a known diameter such as 1/16″ to slide through easily. When the screw is threaded into the anchor, the constriction of the distal open end of the screw as it turns into the threads of the anchor may cause the opening to close down such that a support wire is clamped down by the narrow opening in the screw. The narrow opening may additionally have grip features such as texturing, barbs, ridges, or the like which provide additional connection force to the support wire. In practice, this may allow a surgeon to place an anchor into the bone from one side such as the medial side with the shoulder remaining external to the bone. Then the surgeon may insert the support wire through the opening of the screw as it extends toward the opposite side such as the lateral side. The surgeon may then grab the support wire from the medial side and place a tension on the support wire and then turn the screw with a tool as described above such that the narrow opening clamps down on the support wire. The surgeon may then turn the screw until a desired tension or length has been defined for the support wire. This offers an advantage over current techniques because it allows the surgeon to adjust the length of the support wire gradually in a controlled manner. Furthermore, during any time of the post-op period or later, the surgeon may decide to further adjust the tension or length of the support wire. The surgeon may perform a procedure at that point to simply turn the screw in the desired direction to adjust the performance of the implant. This may be particularly helpful if a change in the anatomical distances occurs such as during growth or decay.

In another embodiment a crimp may be placed on the support wire, not shown in the figures, which is crimped onto the support wire at a known location. This crimp may be placed into a recess on the screw such that it is connected rigidly to the screw. In practice, a crimp may be pre-placed onto the support wire by the manufacture of such a device and the surgeon may adjust the length or tension of the support wire by advancing the screw. Alternatively, the crimp may be placed by the surgeon at the time of surgery to the desired location along the length of the support wire. The surgeon may fixate the crimp to the support wire through any number of manners such as point swaging, rotary swaging, adhesives, welding, soldering, or the like. In this embodiment, the surgeon may apply tension to the support wire and mark a distance to crimp at. The surgeon may then remove the support wire and place a crimp at the desired location using a technique described above.

In another embodiment shown in FIG. 13, the support wire may loop around an anatomical feature within the joint such that both ends are connected to the anchor. For example, the anchor may be placed into the tibia and the support wire may be looped around the fabellar bone on the posterior side of the femur. This is similar to how an MRIT procedure is performed except in the described invention an anchor is included which provides advantages. One end of the support wire may be connected rigidly to one end of the anchor, and the opposite end of the support wire may be connected to a screw as described in the system above. The screw may then be adjusted such that the tension or length of the support wire is adjusted. Alternatively, both ends of the looped support wire may be connected to separate screws which may be adjusted separately within the anchor. Alternatively, one support wire may be connected to a spring element as described below and the other support wire may be connected to a screw which are both contained within the same anchor. Alternatively, two separate anchors may be used in the same bone such as the tibia such that both anchors may exist separately and adjusted separately or have separate termination techniques such as a screw and a spring element. In addition, the connection point for the support wires may be on the anchors but still external to the profile of the bone or configured such that the longitudinal axis of the support wire is not aligned with the longitudinal axis of the anchors. Adjustment and elasticity may be achieved using methods and devices described above.

In another embodiment shown in FIG. 14, a support wire is connected to an extension spring element within the anchor. The extension spring element may provide compliance for the support wire similar to the compliance found in natural joint ligaments. For example, the mid-range of measured axial stiffness of the natural ACL is 1350 lb/in (237 kN/m). The extension spring element may be defined by its spring rate to apply a stiffness to a support wire that is similar to this stiffness. Alternatively, a lower or higher stiffness may be applied to the support wire. In this embodiment, one end of the support wire may be connected rigidly to a structure such as an anchor or a bone or a tissue and the opposite end may be connected to a spring element such that the support wire has an effective stiffness which is lower than the stiffness of the support wire material. This is an advantage over current techniques that either provide limited compliance in the material which may impart impact and other high forces onto the joint, and materials that are intended to have compliance but thereby sacrifice break strength as is the case with nylon suture currently used in MRIT techniques. These materials may indeed have compliance because the material is soft, but therefore may have a lower break strength than the natural ligament and therefore break due to excessive loads. A support wire which has a higher break strength than a natural ligament yet additionally is connected to a spring element that provides compliance offers a significant advantage. Alternatively, the spring element may have a variable spring rate which provides compliance at one level of stiffness for a given distance or force and then applies a second level of stiffness. This may occur by combinations of tension and compression elements and include lower stiffness elements that reach a solid height before imparting force onto elements that have a higher stiffness.

In another embodiment not shown, the extension spring element may be a part of the support wire. For example, certain braid constructions of wire have significantly higher amounts of compliance inherent to their structure than a solid wire of the native material. This is considered ‘construction stretch’ that takes place as the filaments of the braided wire unwind to become straighter. This amount of stretch may be adjusted to meet the desired stiffness requirements of the support wire such that the support wire has the desired compliance which may be close to the natural stiffness of a ligament. In another embodiment, a part or the entire support wire may be comprised of a long extension spring element such as a tight wound extension spring. Such an embodiment may be advantageous because it may provide a longer length for the extension spring element to extend over and therefore higher forces may be achieved.

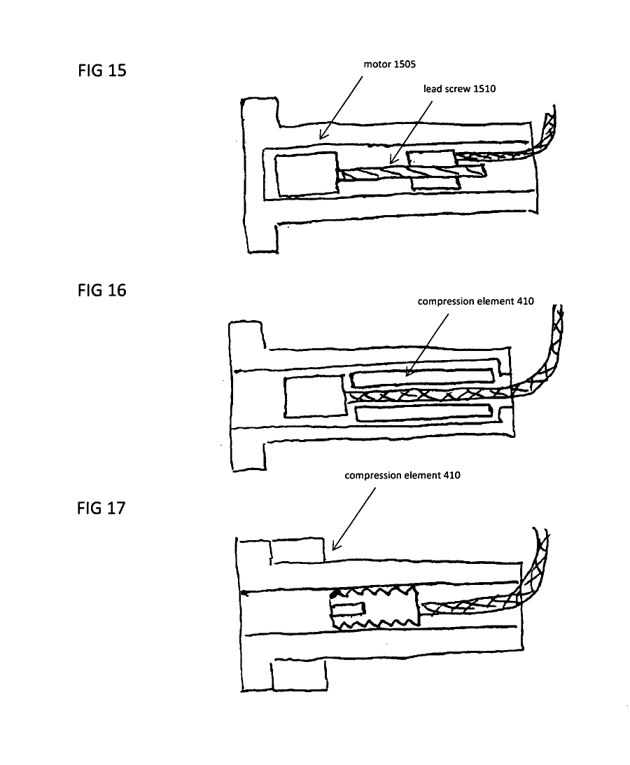

In another embodiment shown in FIG. 16, the spring element may be comprised of a compression element 410. In an exemplary embodiment, the compression element 410 may be a plastic spring comprised of a material such as urethane with a durometer of 60A which has a relatively low compressive modulus of 2000 psi. The compression element 410 may have dimensions of approximately 0.30″ outer diameter, 0.065″ inner diameter, and a 0.75″ free length. Such a urethane spring would have a stiffness of approximately 180 lb/in and a force of approximately 12 lb when compressed to a length of 0.68″, or a 0.070″ change in length. Such a spring may present several advantages over an extension spring element. The stiffness achieved may be much higher for a given size in a compression element 410 and in general there is significantly less risk of an over exertion failure mode. At high loads, compression element's tend to ‘bottom out’ or reach a maximum strain that prevents a more serious failure mode. Additionally, compression elements tend to be cheaper and easier to manufacture than extension spring elements. Alternatively, the compression element 410 may be comprised of other materials such as a compressible plastic with an appropriate compressive modulus. The compression element 410 may be designed to achieve a desired stiffness such as 1350 lb/in or a stiffness which is higher or lower than this amount. Alternatively, the compression element 410 may not be plastic solid spring but instead a disc spring or Belleville washer. Such springs, not shown in the figure, are comprised of a bent washer that may be compressed a relatively short distance for a certain stiffness. However, multiple disc springs can be combined in a variety of orientations to create a desired stiffness, free length, stroke, and force. For example, in one embodiment the compression element 410 may be comprised of a series of 33 disc springs with part number CDM-83204 manufactured by Century Spring. When stacked in a simple accordion pattern, these springs have an approximate stiffness of 215 lb/in and a force of 12.6 lbf at a length of 0.725″, or a 0.054″ change in length. The disc springs may be comprised of stainless steel as illustrated in the numerical example above or in any number of other suitable materials. In FIG. 16, the compression element 410 may be located internal to the anchor such that the support wire has a crimp or other connected component which pulls against the compression element 410. The compression element 410 may be constrained by the opposite wall of the anchor as shown in FIG. 16. The support wire may be routed through the compression element 410 such as the plastic solid spring which has a hollow center or the disc springs which also have a hollow center. One anchor in this embodiment of an implant system may contain the compression element as shown in FIG. 16 and a separate anchor may contain the adjustable features. The surgeon may then adjust the adjustable element until the desired tension or length is achieved in the support wire. In the numerical examples illustrated above a #4-40 screw would be turned approximately 2.8 rotations to achieve a force of 12 lb in the plastic solid spring. This amount of force may be ideal since normal tension loads on the CrCL are estimated at 11 lbf (50N) during walking. The relatively high number of rotations may be ideal since the surgeon will be able to adjust the amount of tension with a higher degree of resolution as compared to a higher pitch system. Alternatively the number of rotations may be lower or higher than given above.

Features may exist which are not shown in the figures which provide indications of the amount of tension or length of the support wire. For example, at the end of the anchor with a compression element 410 a mechanical feature may exist which indicates through the use of a dial or other display method the amount of tension in the support wire. Alternatively, the indication may include electronic components which may communicate to a separate device such as a smart phone or tablet the amount of tension in the support wire. Although not shown, these components could exist within the anchor through the use of strain gauge or alternative mechanism. Alternatively, a separate device could be placed onto the support wire to determine the amount of tension in the support wire and this separate device which is not implanted with the implant system could communicate to any number of other devices or include its own readout display.

In another embodiment shown in FIG. 17, the compression element 410 may alternatively exist externally to the anchor. In an exemplary embodiment shown in the figure the compression element may exist between the shoulder and the bone. In this case, as the tension is increased within the support wire the anchor is pulled into the compression element providing compliance to the implant system in the same manner as described above. An advantage of such an embodiment may be that both the compression element and the adjustable feature exist on the same anchor. In such an embodiment, the support wire may be looped back to the same anchor such that only one drilled hole may be required in the bone. Alternatively, the compression element could be parallel or in some other orientation relative to the adjustable features. For example, the compression element could exist next to the adjustable feature whereby the adjustable feature is connected to the terminal end of one side of the support wire and the compression element is connected to the terminal end of the other side of the support wire. This may be advantageous because both the adjustable feature and the compression element may require long lengths so stacking them in parallel may reduce the size or length of the anchor.

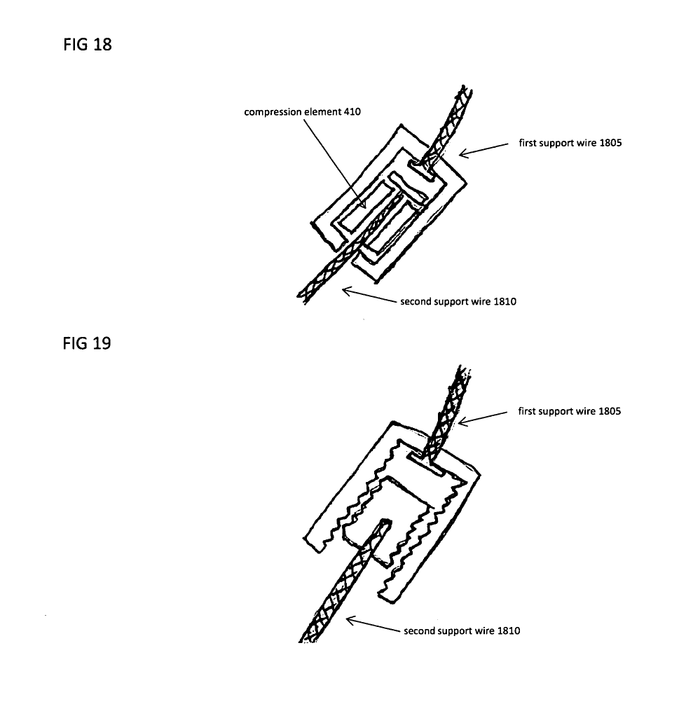

In another embodiment shown in FIG. 18, the compression sprint element may alternatively exist at some point along the length of the support wire. In an exemplary embodiment shown in the figure, the compression element is contained within a shell that is connected to a first support wire and the compression element 410 is compressed by a second support wire. These support wires may be connected such as a loop or may exist as independent wires. As tension is supplied to the support wires, the second support wire imparts a compressive load on the compression element 410 thereby generating a stiffness inherent to the material and size of the compression element 410. This may have the effect of producing a support wire that has an apparent stiffness similar to a natural ligament. Alternatively the stiffness of the support wire may be chosen to be larger or smaller than the stiffness of a natural ligament. In some embodiments the compression element 410 configuration may exist at the back of the joint where it may loop around the fabellar bone. In such an embodiment, the shell of the compression element shown may be shaped be ideally sized for the fabellar bone. This may be advantageous because a larger flatter surface may impart a force to the femur that is distributed over a larger area than a support wire by itself. In some embodiments, the compression element 410 may be a soft plastic piece that routes the support wire around the fabellar bone. In such embodiments, the compression element 410 may not connect a first and second support wire but rather may provide a cushion between the support wire and the bone. The cushion may provide an additional benefit in that it may be compressively compliant and therefore may create an apparent stiffness in the support wire. Alternatively, the cushion may exist at any number of locations between the support wire and the various bones to further define a stiffness in the support wire.

In another embodiment shown in a FIG. 19, a first and second support wire are connected by an adjustable features similar to the screw mechanism described above. In such an embodiment, a second support wire may be connected to a screw and a first support wire may be connected to a shell that has a series of internal threads. The surgeon may turn the screw relative to the shell to create tension or reduce the length of a support wire. This may be advantageous since the assembly described above may be external to a bone such that is may be easier to adjust than an assembly which is contained within a bone. Alternatively, any other number of adjusting mechanisms, such as ratchets, pulleys, linear screws or the like may be used to provide adjustment in the support wire. In some embodiments, an adjustable feature and a compression element 410 may be combined into a single assembly.

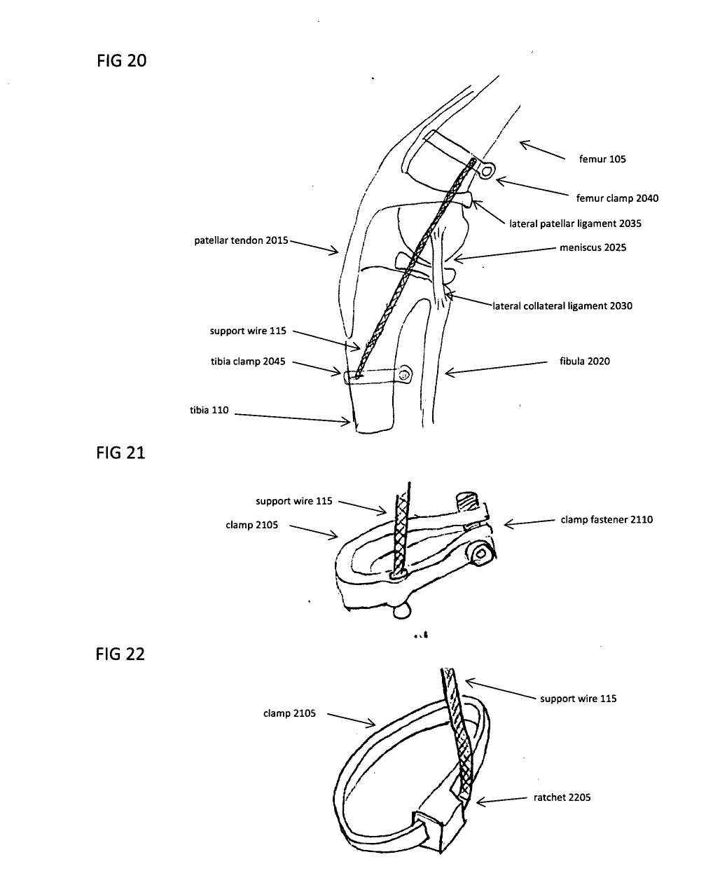

In FIG. 20, an exemplary embodiment of an implant system is shown with a clamping system. From a side view, a stifle joint is appreciated with a femur, tibia and other anatomical structures. Along the femur there exists a femur clamp which is rigidly attached to the femur. Similarly along the tibia there exists a tibia clamp which is rigidly attached to the tibia. In the embodiment shown the tibia clamp and femur clamp do not puncture the bone in the same way as an anchor would. Rather, in the embodiment shown, the clamps are tightly fastened to the bone using compression similar to the operation of a hose clamp or a pipe clamp. In FIG. 20 a support wire is connected to the femur clamp and the tibia clamp. The support wire may provide stability to the joint by resisting motion of the femur posteriorly to the tibia along the tibial plateau and thereby prevent meniscus damage. This is similar to the function of an MRIT or TightRope procedure, however the described invention may be advantageous as a large hole may not be required to be drilled in the bone. Although only one support wire is shown in FIG. 20, it should be appreciated that multiple support wires may exist between the femur clamp and the tibia clamp such as symmetrically opposite on the lateral and medial sides. Furthermore, the support wire may exist entirely on the medial side or the lateral side or alternatively it may exist on both the lateral and medial side as it runs through the stifle joint. The embodiment shown may offer advantages to existing techniques in that is may cause less trauma to the surrounding tissue. This may allow the patient to recovery faster and reduce the amount of time required before a load can be placed on the joint.

In FIGS. 21 and 22, embodiments of a clamping system are shown. In FIG. 21, a clamp which is similar to a pipe clamp is shown. In this embodiment, a clamp is secured to the bone by tightening of a clamp fastener such as a screw.

The clamp may be constructed of any number of materials. In an exemplary embodiment, the clamp may be made of a hard durable material such as a stainless steel type 316LL or the like. Alternatively, the clamp may be constructed of other metallic materials such as titanium, Nitinol, or any other suitable metal. Alternatively, the clamp may be constructed of a non-metallic material such as a PEEK, PTFE, or any other suitable non-metallic material. In some embodiments, the clamp may be constructed of multiple materials. For example, in an exemplary embodiment the base material of the clamp may be a stainless steel with a softer material such as a silicone rubber to provide a cushion as the clamp is fixated to the bone. The clamp may be manufactured through any number of manufacturing methods such as machining, injection molding, or the like. In some embodiments, the clamp or other components of the assembly may be custom made for each patient. In this embodiment, measurements may be taken of the patient's joint before the procedure such as through the use of an MRI, CT, X-ray or the like. These measurements may then be used in the manufacturing of a clamp or other component. For example, selective laser sintering (SLS) may be used to print a stainless steel 316 clamp that can fit the profile of each individual patient's anatomy.

The mechanism for tightening the clamp to the bone may be a screw that is tightened as shown in FIG. 21 to constrict the clamp. Alternatively, a hose clamp mechanism may be used such that one end of the clamp may constricted through a worm screw mechanism. Alternatively, any other number of mechanisms may be used to fixate the clamp to the bone. In some embodiments an adhesive or cement may be used to secure the clamp to the bone. Although only one fastener is shown in FIG. 21, it should be appreciated that multiple fasteners may be used to secure to clamp. In some embodiments, the clamp may exist as two pieces that are screwed together to clamp down onto the bone such as on the medial and lateral sides. Alternatively the two pieces of the clamp in this embodiment may snap together with press fit features or the like. Alternatively, the clamp may exist as a single piece without any fastener such as a snap ring or the like.

In another embodiment shown in FIG. 22, a ratcheting mechanism is used to constrict the clamp to the bone similar to a zip tie mechanism. In this embodiment, the clamp and support wire may be a single piece that has features to ratchet it tightly as the support wire component is tensioned. Alternatively, multiple pieces may be used to create the mechanism shown in FIG. 22. For example, a zip tie mechanism may be tightened to the bone and the end of the zip tie may have a crimp basket that the support wire can connect to. The clamp mechanisms shown in FIG. 21, FIG. 22, and others which may be inferred from the descriptions may be used in combination or separately with one another.

The support wire may be connected to the clamp in any number of ways. In an exemplary embodiment the clamp may have a hole or slot along its rim. The support wire may pass through this hole or slot and then be crimped on the opposite side. Alternatively, there may exist a crimp on the end of the support wire which is placed onto a post which exists on the side of the clamp. This may allow the support wire to pivot with regard to the clamp. Alternatively, the support wire may be rigidly connected to the clamp through any number of methods such as mechanical crimping, welding, or the like. Any other suitable method of connecting a wire to a component.

In some embodiments, the support wire may have a pre-loaded tension to impart a force to the femur and tibia in normal standing or walking conditions. Alternatively, the support wire may not have an initial tension during normal activity and may be implanted to only provide a force under heavy exertion such as running.

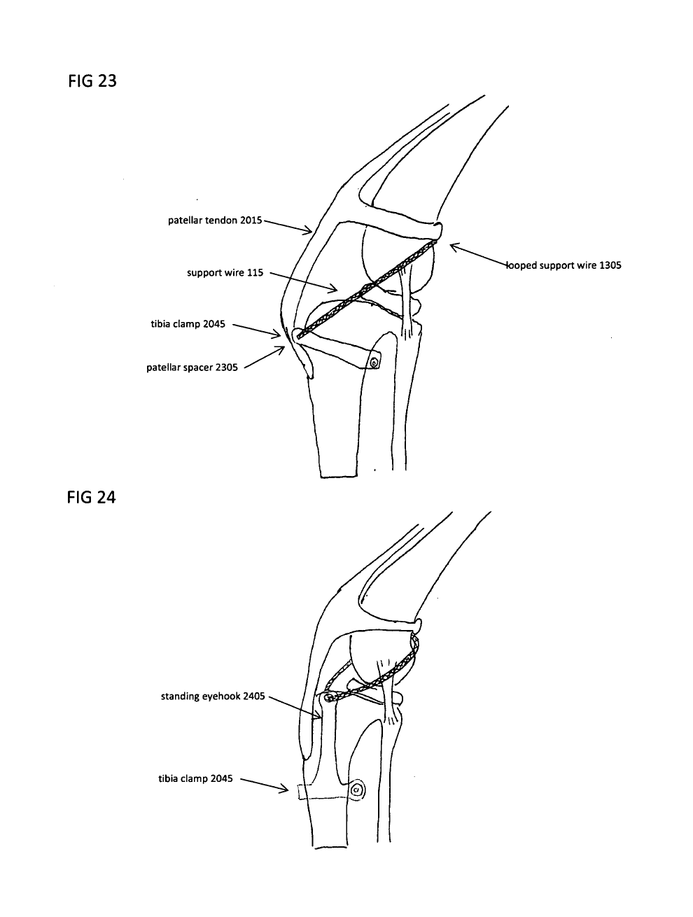

In another embodiment shown in FIG. 23, a clamp may be placed on the tibia that may orient the patellar tendon to a new position. In this embodiment, the anterior portion of the clamp may be positioned such that patellar tendon is moved further anteriorly. This may allow the patellar tendon to put a more anterior oriented force onto the femur by way of the lateral patellar ligament in a similar manner to the function of a TTA procedure where the tibia is advanced anteriorly to shift the patellar tendon. The clamp may have a separate material at the anterior side that pushes on the patellar tendon comprised of a separate material and shape which provides the lease amount of injury to the patellar tendon. In an exemplary embodiment this may be a silicone piece which has a rounded feature to gradually advance the patellar tendon without causing injury. This embodiment may be advantageous over the current techniques because it may be less invasive by not requiring the tibia bone to be cut. Instead a clamp may be simply tightened onto the bone to securely advance the patellar tendon without significantly damaging the bone. In FIG. 23, a support wire is additionally shown that may loop around the fabellar bone similar to certain types of extracapsular procedures. This may provide additional support to the joint in addition to the advancement of the patellar tendon. Although this spacer is only shown at the tibia, it should be appreciated that a similar spacer could be positioned on the femur.

In another embodiment shown in FIG. 24, a clamp may have a standing eyehook feature which may be used for securing the support wire. This may allow the tibia clamp to be positioned lower on the tibia such that it does not affect the patellar tendon but still provide the necessary angle for the support wire to provide the appropriate stability. For example, in certain procedures such as the TightRope, a hole is drilled in the cranial and anterior portion of the tibia such that the angle created in the suture is close to a perpendicular angle. In the embodiment shown, a standing eyehook or a similar features which exists on the clamp may provide an anchor point for a support wire such that support wire is at a similar angle to the final suture in a TightRope procedure. Alternatively, the standing eyehook feature may be used to create an even more perpendicular angle than may be possible by drilling a hole into the tibia.

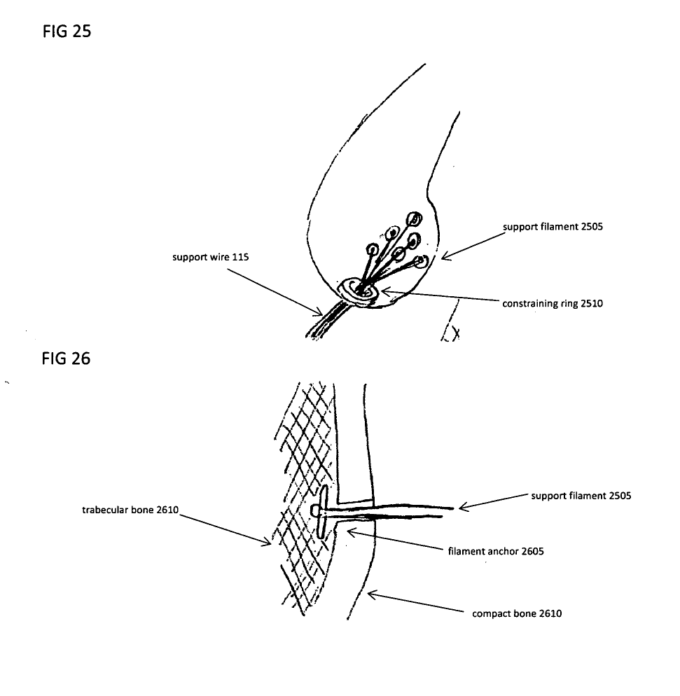

In another embodiment shown in FIG. 25, the support wire may be comprised of a series of support filaments that are anchored separately to the bone. A constraining ring or similar feature may exist to bundle the support filaments together to create the support wire. In an exemplary embodiment, the support wire may be a metallic braided cable such as a 1×7 construction comprised of stainless steel support filaments. The support wire may be 1/16″ in diameter with a strand size of approximately 0.018″ diameter. Alternatively, various constructions may be used such as a 7×7 construction and in this embodiment the support filament may be a braid of 1×7 that comprises the 7×7 support wire. A constraining ring may be created as a crimp on the support wire to prevent the support filaments from unraveling in the braided section while the un-braided section may be able to spread open.

In FIG. 26 a sectional view an individual support filament anchor is shown. In an exemplary embodiment a hole may be drilled through the compact bone and into the trabecular bone such that a support filament may be inserted into the hole. A filament anchor may exist on the end of the support filament which may provide anchoring once inserted into the bone. In an exemplary embodiment this may be a simple toggle bolt which rotates when inserted past the compact bone in the trabecular bone and provides an anchoring force against the inner wall of the compact bone as shown in FIG. 26. Alternatively, any number of other anchoring techniques may be utilized such as suture anchors, bone cement or other adhesives, or the like. Alternatively, the support filament may be looped through one hole in the compact bone and exit through another hole such that a loop is created of the compact bone which provides an anchor for the support filament. The proposed invention may be applied to any bone at any location in the body where anchoring of a cable is beneficial. The described invention may be advantageous over existing anchors and techniques for securing suture because each hole required for a support filament may be significantly smaller than a hole required for the entire support wire or suture. Therefore, the trauma may be less. Additionally, an advantage of the proposed invention is that if an individual anchor or filament breaks, there is redundancy in the system through the other support filaments.

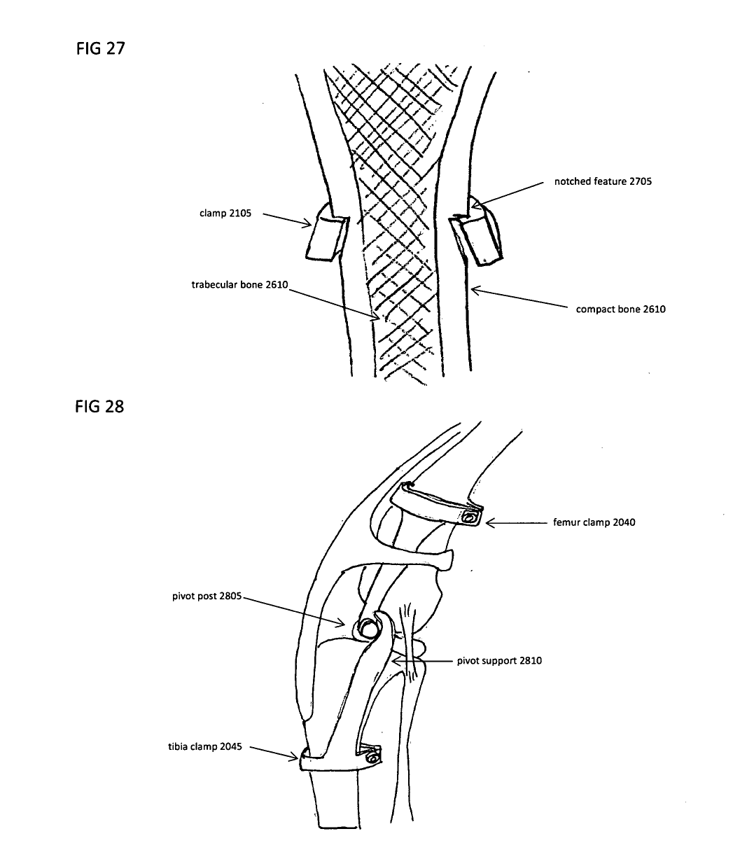

In another embodiment shown in FIG. 27, a clamp is shown on a bone with a notched feature in the bone. In this embodiment, a notch feature may be created on the surface of the bone into the compact bone such that an overhang or lip exists. Then a clamp may be placed onto the bone underneath the notched feature such that an upward force on the clamp is counteracted by the notched feature. In an exemplary embodiment, the notched feature may be created with a surgical tool for grinding or cutting bone. The notched feature may exist only a relatively small amount such as 1/16″ into the compact bone causing minimal trauma to the surrounding tissue. The clamp may then be placed onto the bone under the notched feature. Additional securing features may be used such as fasteners, bone cements, or the like. A support wire that provides upward force may hold the clamp securely in place against the notched feature. The proposed invention may be advantageous over other anchoring techniques because it may cause less trauma by only cutting a minimal amount into only the compact bone.

In another embodiment shown in FIG. 28, a femur clamp and tibia clamp are shown with a pivot post and pivot support. The pivot post and pivot support may exist away from the clamping location of the clamps such they are at or near the virtual pivot location of the joint on the medial or lateral side of the joint. In an exemplary embodiment, the pivot post may be a boss feature which extends laterally or medially away from the joint. The pivot support may provide a feature for the pivot post which prevents posterior movement but allows rotation and any other movement such that the joint has full mobility. In this embodiment, the pivot post may prevent the femur from sliding posteriorly down the slope of the tibia plateau and onto the meniscus because the pivot support may provide an anterior force. This may be similar to the function of a brace which is worn externally to provide stability to the joint. The pivot post and pivot support may be any number of features which allow for rotation but prevent movement such as any number of hinge or pivot mechanisms such as a butt hinge, t-hinge, strap hinge, or the like. The proposed invention may be advantageous over existing techniques in that is may provide the stability of a brace to a joint without the need to wear an external device.

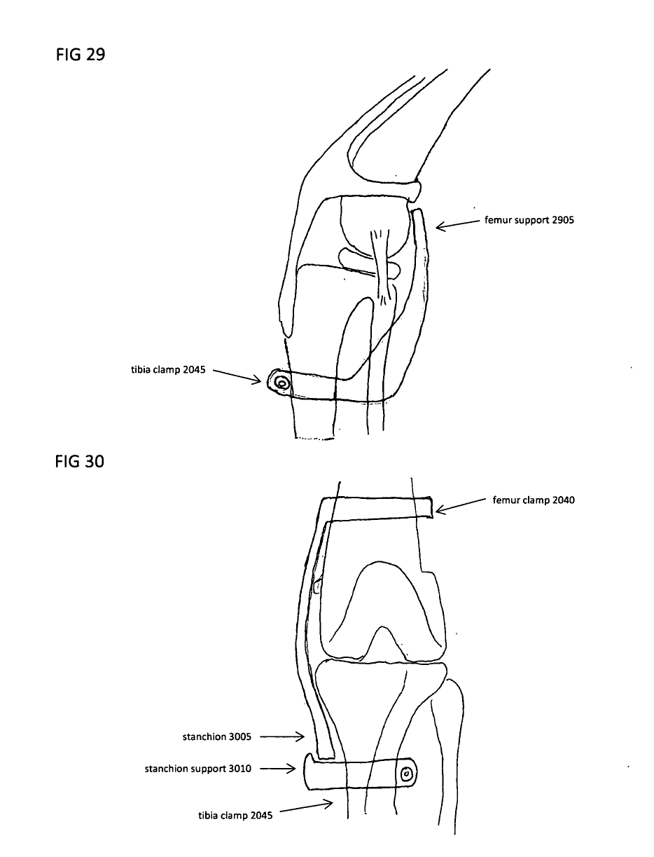

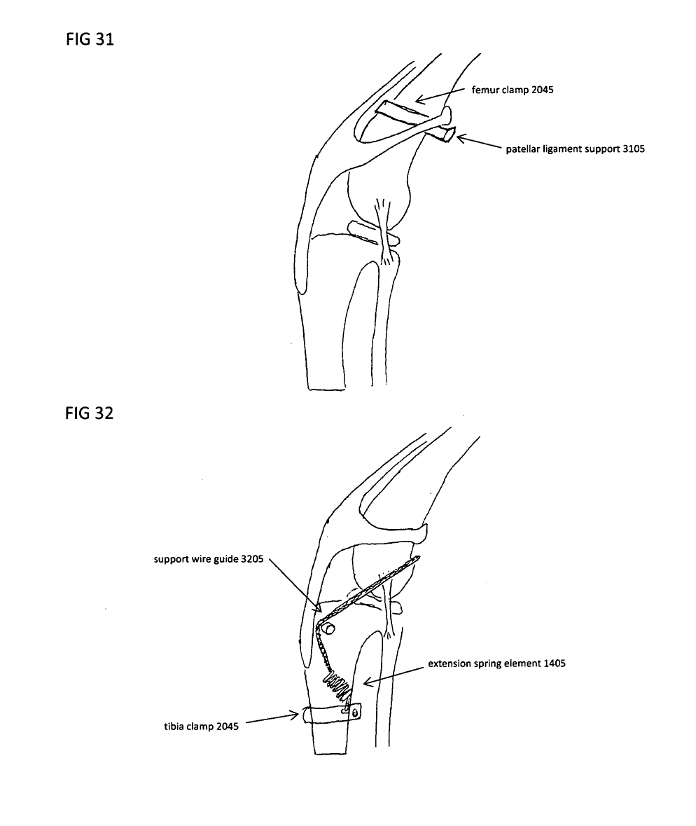

In another embodiment shown in FIG. 29, a tibia clamp may have a femur support feature which extends cranially. The femur support may be connected to the tibia clamp and then extend posteriorly and cranially such that is rests posterior to the femur as shown in FIG. 29. The femur support may provide a hard stop along the posterior section of the femur that prevents the femur from moving posteriorly and thereby damaging the meniscus but still may allow for the femur to rotate in a natural manner. In an exemplary embodiment, femur support may be constructed from a rigid material such as a stainless steel that can sustain the necessary force to maintain the position of the femur. Furthermore the femur support may have a softer material such as a silicone rubber on the surface which contacts the femur such that minimal injury is created. In another embodiment not shown, a spacer may be placed between the head of the femur and the head of the tibia at or near the location of the meniscus which prevents the femur from impinging on the meniscus. This spacer may be comprised of any number of materials and shapes to prevent the femur from impinging on the meniscus. For example, the spacer may be a silicone rubber which provides vertical stability to the femur similar to the meniscus which reduces the force on the meniscus if the femur slides posteriorly. Alternatively, the spacer may be balloon or disc that can be inflated with a viscous material once inserted into the joint space.

In another embodiment shown in FIG. 30, a stanchion and stanchion support are connected to a tibia clamp and a femur clamp. The stanchion extends down from the femur clamp to a location lower than the joint space. The stanchion support extends outward from the tibia clamp such that it is directly below the stanchion. When a force is placed on the femur that may cause the femur to impinge on the meniscus, the stanchion may also move downward and contact the stanchion support. This may prevent the femur from significantly impinging the meniscus such that the load of the femur is transmitted through the stanchion and stanchion support. The interface between the stanchion and stanchion support may be curved such that the femur can rotate freely and the gap between the stanchion and stanchion support is maintained. Any number of materials may be used at this interface to dampen the force between the femur and tibia including the use of soft biocompatible rubbers such as silicone or PTFE. Alternatively, the stanchion may exist on the tibia clamp and the stanchion support may exist on the femur clamp. Alternatively, both the femur clamp and the tibia clamp may have similar features which extend and interface at some location between the tibia clamp and femur clamp such as at the virtual pivot axis of the joint. The proposed invention may be advantageous because it may allow for the femur to be supported and minimize meniscus crushing at a location that is away from the joint.

In another embodiment shown in FIG. 32, a patellar ligament support is shown on the femur clamp. The patellar ligament support may be placed under the lateral patellar ligament such that the ligament is held at a higher location on the femur. This may orient the angle of the lateral patellar ligament such that more tension is placed on the lateral patellar ligament in an orientation that pulls the femur anteriorly to prevent the femur from moving posteriorly down the tibia plateau. The construction may be similar to the patellar spacer described in FIG. 23. The proposed invention may be advantageous because it may cause less trauma than current surgical procedures for altering the angle of force in the patellar ligament such as the TTA procedure.

In another embodiment shown in FIG. 32, a support wire guide is shown with an extension spring element. The support wire guide may be affixed to the tibia or the femur such that the support wire provides an appropriate angle across the joint while the anchored location of the support wire is much lower along the length of the tibia. In an exemplary embodiment, the tibia clamp may provide the anchoring of the support wire but the location may be caudal along the length of the tibia such that it does not affect the patellar tendon. The support wire guide then may route the support wire such that it is angled adequately across the joint to prevent the femur from sliding posteriorly. The support wire guide may be fixed to the bone through any number of techniques such as drilling a hole and inserting a pin, using bone cement to secure the guide, screwing in an anchored post, or the like. Additionally, an extension spring element may exist that provides compliance to the support wire. The extension spring element may be an extension spring as shown in FIG. 32 with a desired stiffness to allow the joint to move properly. Alternatively the spring element may be any number of other compliant structures including those described above such as compression element 410. The proposed invention may be advantageous because it may secure the support wire sufficiently but at a location that is further away from the joint while the support wire is still routed at an appropriate angle.

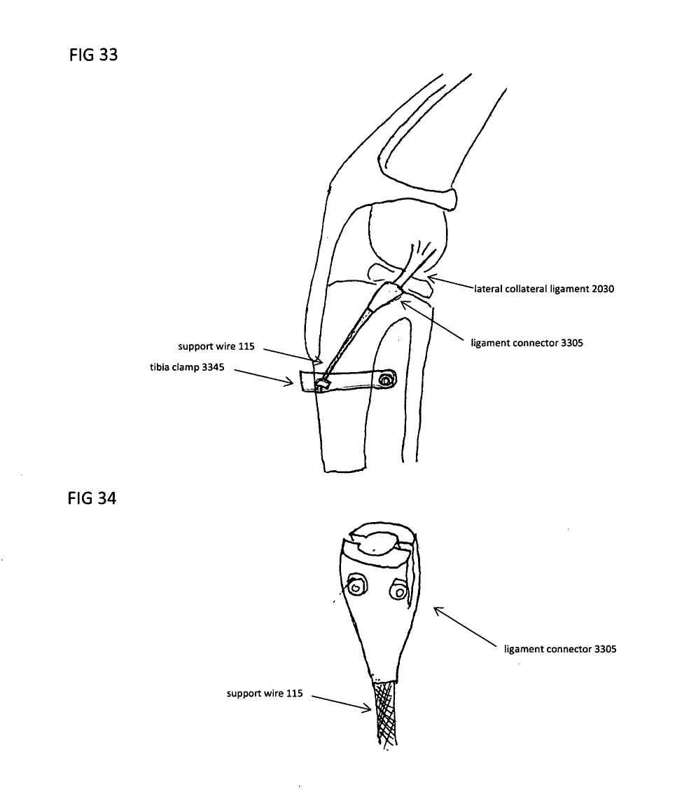

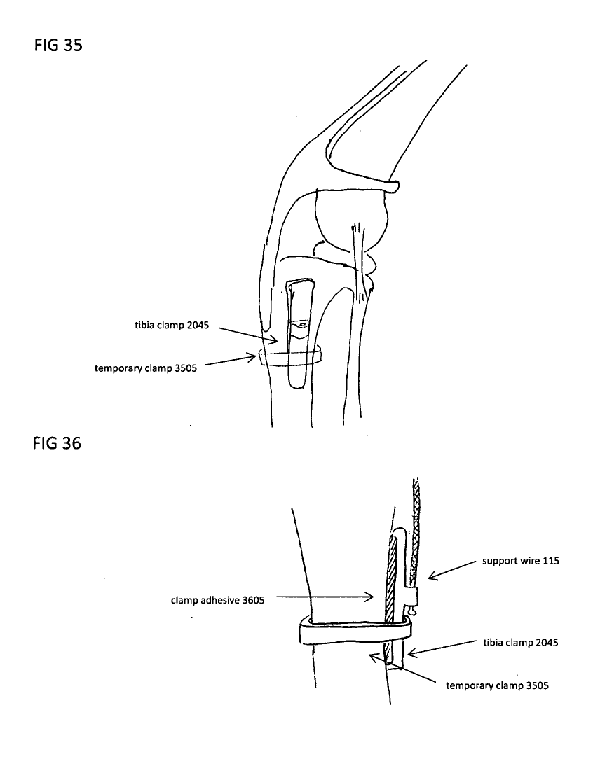

In another embodiment shown in FIG. 33, a ligament connector is connected to a ligament and a support wire. The ligament connector may be used to secure an existing ligament in the joint such as the lateral collateral ligament (LCL). The other end of the ligament connect may have a support wire which can provide tension to another body connected to a rigid structure. In an exemplary embodiment, the LCL may be connected to the ligament connector through manners described below and then the support wire may be connected to a tibia clamp at an anterior location. The LCL may then provide sufficient stability across the joint similar to an MRIT or TightRope procedure. The ligament connector is shown in more detail in FIG. 34. In an exemplary embodiment, the ligament connector may be a single piece of material with a hole drilled in the center, two slots on each side of the hole, and a series of threaded holes for fasteners. The fasteners may be tightened to clamp down on the hole and deform the connector such that a ligament which is placed in the hole would be securely clamped. Alternatively, any number of other mechanisms for securing a ligament to a connector may be used such as wrapping a ligament around a threaded post that is tightened, thermal bonding, adhesive bonding, or the like. The proposed invention may be advantageous because the LCL is already connected to the femur so no additional connection is required at this location. In an alternative embodiment not shown, there may be two ligament connectors with one on each end of the support wire. One end may be connected to a ligament on the femur such as the LCL and the other may be connected to a ligament or tendon on the tibia such as a part of or the entirety of the patellar tendon. In this embodiment, the support wire may carry the tension between the two ligaments or tendons which are already anchored to the bones. This may be advantageous because no additional anchoring or clamping to the bone may be required which may minimize the amount of injury. Alternatively, the device may be connected to a single ligament such that tension is carried from one end of the ligament to the other through the ligament connectors and support wire. In this embodiment, a partially torn or weak ligament may be strengthened by the addition of such a device that uses the existing ligament to maintain joint stability.

In another embodiment not shown, a sleeve material may be placed on a ligament in the joint which provides increased strength to the ligament. In an exemplary embodiment the sleeve material may be a heat shrink material such as a PTFE, FEP, PET or the like which may be placed onto the ligament and then secured with the use of heat, mechanical swaging or the like. This may add strength to the ligament and prevent further injury or degradation of the ligament.

In another embodiment shown in FIG. 34, a tibia clamp is secured to the tibia with a temporary clamp. The temporary clamp may be used hold the tibia clamp to the tibia for some post operative period. In an exemplary embodiment, the tibia clamp may be secured to the tibia with the use of bone cements or other adhesives that may have extended curing times. This may cause the tibia clamp to not be fully attached to the tibia for a period of time post operatively that prevents the patient from placing a significant load on the joint. The temporary clamp may provide additional securement of the tibia clamp to the tibia during this period. The temporary clamp may be comprised of a biodegradable material such as a poly-lactic acid (PLA) or poly-lactic co-glycolic acid (PLGA) which dissolves over a period of time post operatively after providing the necessary clamping to the tibia clamp. Alternatively, the temporary clamp may be constructed of a non-degradable material such as a stainless steel which may be removed once the tibia clamp is secured through other means. In FIG. 36, the temporary clamp is shown around the tibia to secure the tibia clamp with a clamp adhesive at the bone interface. The proposed invention may be advantageous because it may provide support to the tibia clamp during the post operative period.

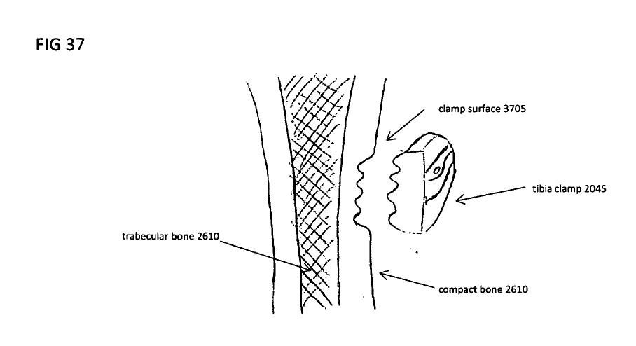

In another embodiment shown in FIG. 37, a tibia clamp is shown secured to a tibia along a clamp surface. The clamp surface may be cut or shaped into the compact bone to have features which provide an improved surface for a tibia clamp. In an exemplary embodiment, the clamp surface may be an undulating surface that matches a surface on the tibia clamp. The tibia clamp may be secured to the tibia using a bone cement or other clamp adhesive. The clamp surface may be designed to provide an increased surface area along the bone for a given anatomical location. Alternatively, the clamp surface may be any number of shapes or profiles that improves the ability to secure the tibia clamp to the tibia. The proposed invention may be advantageous because it may allow the tibia clamp to be securely attached to the tibia with minimal injury.

In some embodiments described above, the operation to implant the device may take place at various times. In an exemplary embodiment, the device may be secured to the bone or bones during one operation and allowed to heal partially or completely before a second operation is performed to implement the mechanism which provides stability. Alternatively, the device may be implanted in a second joint while an operation is being performed on a first joint. The device may be used to provide stability in joints that have not yet torn or injured the CCL, but where it may be suspected that injury could occur in the future. This may prevent injury from occurring.

Although embodiments of various methods and devices are described herein in detail with reference to certain versions, it should be appreciated that other versions, embodiments, methods of use, and combinations thereof are also possible. Therefore the spirit and scope of the invention should not be limited to the description of the embodiments contained herein. Furthermore, although the various embodiments and description may specify certain anatomical locations, species, or surgical procedures, it should be appreciated that these embodiments apply to other locations, species, and surgical procedures.

What is claimed is:

1. An apparatus for improving the stability of a joint comprised of:
a. a securing anchor configured to rigidly attach to a bone with at least a portion of the anchor contained within the profile of the bone and at least a portion of the anchor external to the profile of the bone;
b. a linking element connected to said anchor wherein the point of connection is on the portion of the anchor external to the profile of the bone.

2. An apparatus for improving the stability of a joint whereby the apparatus is comprised of:
a. a securing anchor configured to rigidly attach to a bone with at least a portion of the anchor contained within the profile of the bone forming a longitudinal axis of the anchor;
b. a linking element connected to said anchor wherein at the point of connection the linking element is not substantially aligned with the longitudinal axis of said anchor.

3. The apparatus of claim 1 wherein the apparatus further includes an elastic element with a predetermined stiffness wherein the elastic element is configured to receive a compressive load when a tensile load is applied to the linking element.

4. The apparatus of claim 1 wherein the apparatus further includes an adjustment mechanism for adjusting the tensile load in the linking element wherein the adjustment mechanism is a screw mechanism attached to the linking element and the securing anchor.

5. The apparatus of claim 2 wherein the linking element is a flexible cable.

6. The apparatus of claim 5 wherein the flexible cable is connected to the anchor with a crimped area which is larger than the nominal diameter of the cable.

7. The apparatus of claim 2 wherein the apparatus further includes an elastic element with a predetermined stiffness.

8. The apparatus of claim 7 wherein the predetermined stiffness is between one third and three times the stiffness of a natural ligament.

9. The apparatus of claim 7 wherein the elastic element is configured to receive a compressive load when a tensile load is applied to the linking element.

10. The apparatus of claim 9 wherein the elastic element is comprised of an elastomeric material with a stiffness dependent on the compressive modulus of elasticity of the material.

11. The apparatus of claim 7 wherein the elastic element is the securing anchor wherein the anchor is configured to flex when a tensile load is placed on the linking element.

12. The apparatus of claim 7 wherein the elastic element is the linking element wherein the linking element is configured to stretch when a tensile load is placed upon the linking element.

13. The apparatus of claim 2 wherein the apparatus further includes an adjustment mechanism for adjusting the tensile load in the linking element

14. The apparatus of claim 13 wherein the adjustment mechanism is a screw mechanism attached to the linking element and the securing anchor.

15. The apparatus of claim 14 wherein the screw mechanism is substantially in line with the longitudinal axis of the linking element.

16. The apparatus of claim 2 wherein the securing anchor is connected to the femur bone and the linking element is also connected to a second securing anchor which is rigidly attached to the tibia bone.

17. A system for improving the stability of a canine stifle joint comprised of:
a. a first securing anchor attached to a femur bone
b. a second securing anchor attached to a tibia bone
c. a flexible cable connected to both the first and second anchors
d. wherein at least one connection point includes a compressive element that creates an apparent elasticity of the flexible cable which is between one third and three times the elasticity of a natural ligament when placed in tension.
e. wherein the apparatus further includes at least one adjustment mechanism which is configured to adjust the tensile load in the flexible cable between the first and second anchor

18. The system of claim 17 wherein the first anchor contains a connecting feature external to the profile of the femur configured to received the support wire and further configured to include the compressive element.

19. The system of claim 17 wherein the second anchor contains the adjustment mechanism comprised of an internal thread and a wire screw connected to the flexible cable further configured to engage with the internal thread of the second anchor.
